(19) United States
(12) Patent Application Publication
(10) Pub. No.: US 2009/0216273 A1
(43) Pub. Date: Aug. 27, 2009

(54) CURVED FACET JOINT FIXATION ASSEMBLY AND ASSOCIATED IMPLANTATION TOOL AND METHOD

(75) Inventor: John Cox, Acworth, GA (US)

Correspondence Address: Clements Bernard PLLC, 1901 Roxborough Road, Suite 300, Charlotte, NC, 28211, US

(73) Assignee: U. S. SPINAL TECHNOLOGIES, L.L.C., Boca Raton, FL (US)

(21) Appl. No.: 12/388,959

(22) Filed: Feb. 19, 2009

Related U.S. Application Data
(60) Provisional application No. 61/029,618, filed on Feb. 19, 2008.

(51) Int. Cl. A61B 17/70; A61B 17/88

(52) U.S. Cl. 606/246; 606/264; 606/254; 606/279

(57) ABSTRACT

The present disclosure relates to a curved facet joint fixation assembly and an associated implantation tool and method. The curved facet joint fixation assembly includes an elongated curved shaft adapted to conform to various spinal morphologies. The implantation tool includes a bone fastening device with an angled lower end portion and a removable drive for cutting a hole and for driving the curved facet joint fixation assembly through the hole.

### CROSS-REFERENCE TO RELATED APPLICATION(S)

The present non-provisional patent application claims the benefit of priority of U.S. Provisional Patent Application No. 61/029,618, filed on Feb. 19, 2008, and entitled “CURVED FACET JOINT FIXATION ASSEMBLY,” the contents of which are incorporated in full by reference herein.

### FIELD OF THE INVENTION

The present invention relates generally to surgical assemblies, tools, and methods for performing bone arthrodesis. More specifically, the present invention relates to a curved facet joint fixation assembly and an associated bone fastening device and method.

### BACKGROUND OF THE INVENTION

Bone arthrodesis, or fusion, is a surgical procedure that is used to stabilize or immobilize impaired bones or joints such that they can heal. More specifically, facet arthrodesis is a surgical procedure that is used to stabilize or immobilize a spinal facet joint in the treatment of an injury or degenerative condition. Conventional facet arthrodesis systems and methods utilize bone screws that are driven through the superior and inferior facets so as to allow the adjoined bone sections to fuse together. Conventional facet arthrodesis systems and methods also utilize wires that are looped around the superior and inferior facets so as to allow the adjoined bone sections to fuse together. The surgical procedures that must be employed to implant these bone screws or wires are difficult and time consuming. Therefore, there is a need for improved surgical assemblies, tools, and methods for performing bone arthrodesis.

U.S. patent application Ser. No. 10/683,076 (U.S. Patent Application Publication No. 2004/0143268), Ser. No. 10/973,524 (U.S. Patent Application Publication No. 2005/0234459), and Ser. No. 12/122,498 (not yet published) (Falahee et al.) disclose a conventional system for performing bone arthrodesis that includes an implant for bone arthrodesis and a bone fastening device. The implant includes a fastener with an elongated shaft having a head at one end and a bone piercing point at the opposite end. A first washer has a structure for engaging the head of the shaft so as to be polyaxially pivotable with respect to the head. A locking member has a structure for engaging the shaft. The locking member can have a second washer pivotally engaged thereto. The bone fastening device can include an elongated cannula with a collet for detachably engaging the first washer and for advancing the first washer. A structure is provided for engaging the fastener and for advancing and rotating the fastener through the collet and through the first washer. The bone arthrodesis device further includes a lower end portion extending from the cannula. The lower end portion has structure for detachably engaging the locking member. The fastener, first washer, and locking member are aligned such that the advancing fastener will advance through the first washer, drill through the bone, and move into the locking member. A method for performing bone arthrodesis is also disclosed. Although an improvement, this facet arthrodesis system is difficult to use with various spinal morphologies. Therefore, there is still a need for improved surgical assemblies, tools, and methods for performing bone arthrodesis.

### BRIEF SUMMARY OF THE INVENTION

In one exemplary embodiment, the present invention provides a curved joint fixation assembly including a curved elongated shaft with a head and an end; and a washer having a structure for engaging the end; wherein the curved elongated shaft is curved based on a spinal morphology of a receiving patient. The curved joint fixation assembly further includes a plurality of ridges on the curved elongated shaft; and a pawl on the washer for engaging the plurality of ridges. The head optionally includes a hexagonal opening connected to a removable drive of a bone fastening device. The end can include a rounded surface. The curved joint fixation assembly further includes a plurality of serrations on the washer for engaging a bone surface. Optionally, the curved elongated shaft is disposed to a removable drive of a bone fastening device, and wherein the washer is disposed to a lower end portion of the bone fastening device. The bone fastening device includes a cutting tip to bore a hole in a joint and a driving tip to drive and lock the curved joint fixation assembly in the hole.

In another exemplary embodiment, the present invention provides a curved joint fixation assembly implantation tool including an elongated housing terminating in a lower end portion, wherein the lower end portion is at an angle in relation to the elongated housing; a removable drive disposed within the elongated housing, wherein the removable drive terminates with one of a cutting tip and a driving tip; and a curved joint fixation assembly with a curved elongated shaft disposed to the driving tip and a washer disposed to the lower end portion. The curved elongated shaft is curved based on a spinal morphology of a receiving patient. The removable drive includes a flexible tip adapted to fit within an opening from the elongated housing to the lower end portion. The flexible tip can include a coating of a flexible material. The curved joint fixation assembly implantation tool can further include a main body disposed to the elongated housing; wherein the main body includes a handle and one or more triggers. Optionally, the curved joint fixation assembly implantation tool further includes an opening in the main body for providing torque to a drive shaft in the removable drive. The removable drive includes a first removable drive with a cutting tip, and a second removable drive with a driving tip with the curved joint fixation assembly. The first removable drive is utilized to bore a hole in a joint and removed from the elongated housing and replaced with the second removable drive to drive in the curved joint fixation assembly.

In a further exemplary embodiment, the present invention provides a method of implanting a curved joint fixation assembly including boring a hole in a joint with a cutting tip; removing the cutting tip; positioning a driving tip with a curved joint fixation assembly; driving in the curved joint fixation assembly; and locking the curved joint fixation assembly. The method can further include determining a spinal morphology of a receiving patient; and selecting a curvature of the curved joint fixation assembly responsive to the spinal morphology. The method is performed by a bone fastening device. Optionally, the bone fastening device includes an elongated housing terminating in a lower end portion, wherein the lower end portion is at an angle in relation to the elongated housing; a removable drive disposed within the elongated housing, wherein the removable drive terminates with one of the cutting tip and the driving tip; and a curved joint fixation assembly with a curved elongated shaft disposed to the driving tip and a washer disposed to the lower end portion. The method can further include locking the bone fastening device to the joint; and performing the removing and positioning steps while the bone fastening device is locked to the joint.

### BRIEF DESCRIPTION OF THE DRAWINGS

The present invention is illustrated and described herein with reference to the various drawings, in which like reference numbers are used to denote like assembly or tool components/method steps, as appropriate, and in which:

FIG. 1a is a perspective view of a conventional tool for implanting a facet joint fixation assembly;

FIG. 1b is another perspective view of the tool for implanting a facet joint fixation assembly of FIG. 1, highlighting the use of a protective cannula or sheath;

FIG. 2 is a side planar view of a conventional facet joint fixation assembly;

FIG. 3 is a perspective view of a portion of the facet joint fixation assembly of FIG. 2;

FIG. 4 is another perspective view of a portion of the facet joint fixation assembly of FIG. 2;

FIG. 5 is a side planar view of a portion of the facet joint fixation assembly of FIG. 2;

FIG. 6 is a further perspective view of a portion of the facet joint fixation assembly of FIG. 2;

FIG. 7 is a partially transparent perspective view of a portion of the facet joint fixation assembly of FIG. 2;

FIG. 8 is a still further perspective view of a portion of the facet joint fixation assembly of FIG. 2;

FIG. 9 is a still further perspective view of a portion of the facet joint fixation assembly of FIG. 2;

FIG. 10 is a still further perspective view of a portion of the facet joint fixation assembly of FIG. 2;

FIG. 11 is a partially cut away perspective view of a portion of the facet joint fixation assembly of FIG. 2;

FIG. 12a is a cross-sectional side view illustrating the manner of engagement between the fastener, the first washer and second washer, and the locking member of the facet joint fixation assembly of FIG. 2;

FIG. 12b is another cross-sectional side view illustrating the manner of engagement between the fastener, the first washer and second washer, and the locking member of the facet joint fixation assembly of FIG. 2;

FIG. 12c is a further cross-sectional side view illustrating the manner of engagement between the fastener, the first washer and second washer, and the locking member of the facet joint fixation assembly of FIG. 2;

FIG. 13 is a perspective view illustrating a collet associated with the tool for implanting a facet joint fixation assembly of FIG. 1;

FIG. 14 is a planar side view illustrating the deployment of the facet joint fixation assembly of FIG. 2 using the tool for implanting a facet joint fixation assembly of FIG. 1;

FIG. 15 is another planar side view illustrating the deployment of the facet joint fixation assembly of FIG. 2 using the tool for implanting a facet joint fixation assembly of FIG. 1;

FIG. 16 is a cross-sectional side view illustrating the deployment of the facet joint fixation assembly of FIG. 2 using the tool for implanting a facet joint fixation assembly of FIG. 1;

FIG. 17 is a perspective view illustrating the deployment of the facet joint fixation assembly of FIG. 2 using the tool for implanting a facet joint fixation assembly of FIG. 1;

FIG. 18 is another perspective view illustrating the deployment of the facet joint fixation assembly of FIG. 2 using the tool for implanting a facet joint fixation assembly of FIG. 1;

FIG. 19 is a further perspective view illustrating the deployment of the facet joint fixation assembly of FIG. 2 using the tool for implanting a facet joint fixation assembly of FIG. 1;

FIG. 20 is a still further perspective view illustrating the deployment of the facet joint fixation assembly of FIG. 2 using the tool for implanting a facet joint fixation assembly of FIG. 1;

FIG. 21 is another cross-sectional side view illustrating the deployment of the facet joint fixation assembly of FIG. 2 using the tool for implanting a facet joint fixation assembly of FIG. 1;

FIG. 22 is a further cross-sectional side view illustrating the deployment of the facet joint fixation assembly of FIG. 2 using the tool for implanting a facet joint fixation assembly of FIG. 1;

FIG. 23 is a still further perspective view illustrating the deployment of the facet joint fixation assembly of FIG. 2 using the tool for implanting a facet joint fixation assembly of FIG. 1;

FIG. 24 is a cross-sectional side view illustrating both the facet joint fixation assembly of FIG. 2 and the tool for implanting a facet joint fixation assembly of FIG. 1;

FIG. 25 is another cross-sectional side view illustrating both the facet joint fixation assembly of FIG. 2 and the tool for implanting a facet joint fixation assembly of FIG. 1;

FIG. 26 is a further cross-sectional side view illustrating both the facet joint fixation assembly of FIG. 2 and the tool for implanting a facet joint fixation assembly of FIG. 1;

FIG. 27 is a perspective view of a bone fastening device for implantation of a curved facet joint fixation assembly according to an exemplary embodiment of the present invention;

FIG. 28 is a perspective view of a removable drive with a bolt attached to a drive shaft for the bone fastening device of FIG. 27 according to an exemplary embodiment of the present invention;

FIG. 29 is a perspective view of a removable drive with a drill head attached to a drive shaft for the bone fastening device of FIG. 27 according to an exemplary embodiment of the present invention;

FIG. 30 is a perspective view of an elongated housing with the removable drive of FIGS. 28 and 29 removed according to an exemplary embodiment of the present invention;

FIG. 31 is a perspective view of the bone fastening device of FIG. 27 with the removable drive of FIGS. 28 and 29 removed according to an exemplary embodiment of the present invention;

FIG. 32 is a top view of a tip of the removable drive of FIGS. 28 and 29 with a chisel tip bit according to an exemplary embodiment of the present invention;

FIG. 33 is a cross-sectional view of the lower end portion of the bone fastening device of FIG. 27 according to an exemplary embodiment of the present invention;

FIGS. 34-36 are various perspective views of a curved facet joint fixation assembly for implantation according to an exemplary embodiment of the present invention; and

FIG. 37 is a flowchart of a curved facet joint fixation assembly implantation mechanism according to an exemplary embodiment of the present invention.

### DETAILED DESCRIPTION OF THE INVENTION

In various exemplary embodiments, the present invention provides a curved facet joint fixation assembly and an associated bone fastening device and method for implanting the assembly. As described above, U.S. patent application Ser. No. 10/683,076 (U.S. Patent Application Publication No. 2004/0143268), Ser. No. 10/973,524 (U.S. Patent Application Publication No. 2005/0234459), and Ser. No. 12/122,498 (not yet published) (Falahee et al.) disclose a conventional system for performing bone arthrodesis that includes an implant for bone arthrodesis and a bone fastening device. Advantageously, the curved facet joint fixation assembly of the present invention is better suited for various spinal morphologies.

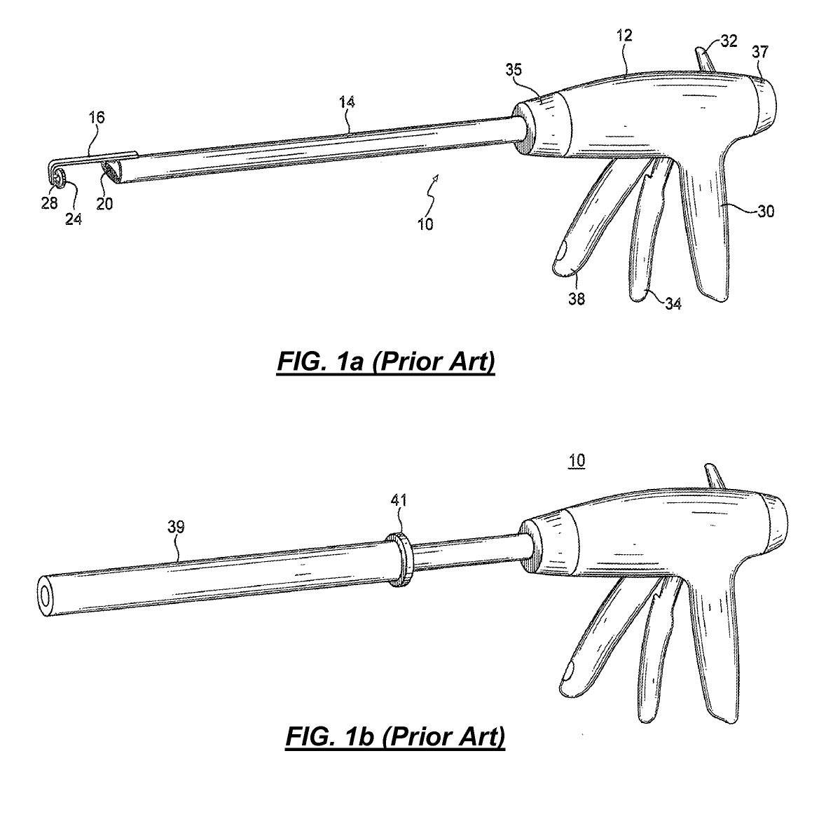

Referring to FIG. 1a, a bone fastening device 10 has a main body 12 and an elongated housing 14 terminating in a lower end portion 16. The device 10 has a structure for holding a first washer 20 at a distal portion of the elongated housing 14 and a second washer 24 and locking member 28 in the lower end portion 16. A handle 30 is provided to grip the device 10, and triggers 34 and 38 can be provided to operate the device 10 during the implantation process. A guide knob 35 can be operated to rotate the housing 14 and attached lower end portion 16 to properly position the second washer 24. A locking lever 32 can be provided to lock the device 10 on the bone or joint after the first washer 20 and second washer 24 have been properly positioned. The locking lever 32 can be unlocked to allow repositioning of the first washer 20 and second washer 24. A knob 37 can be provided to manually advance the fastener and apply appropriate torque.

A slidable protective cannula or sheath can be used to facilitate insertion of the device 10 into the body and cover lower end portion 16. Referring to FIG. 1b, there is illustrated a bone fastening device 10 having a protective cannula or sleeve 39 for shielding the housing 14 and lower end portion 16 during the insertion process. A grip 41 can be used to pull back the cannula 39 prior to use. The housing 14, lower end portion 16, and guide knob 35 can be detachable from the main body 12. Another housing 14, lower end portion 16, and guide knob 35, with another implant, can be attached for reuse of the main body 12.

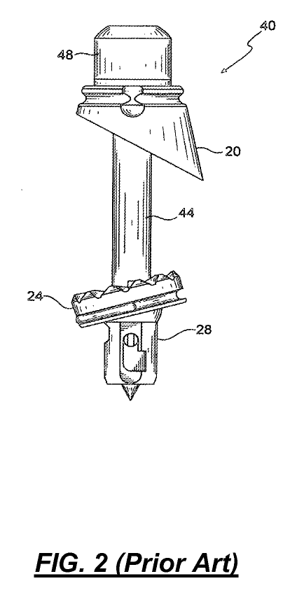

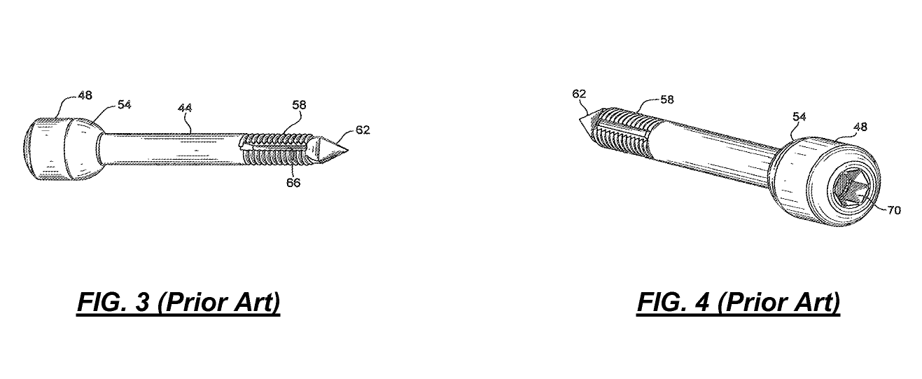

Referring to FIG. 2, the implant 40 includes a fastener having an elongated shaft 44 and a head 48. The fastener engages the first washer 20 and second washer 24 and locking member 28. As illustrated in FIGS. 3 and 4, the head 48 can have a convex surface 54 for use in engaging the first washer 20 as is described in greater detail herein. Threads 58 are provided on the shaft 44 for the purpose of engaging the locking member 28. Other engagement structures can also be used. A pointed end 62 is provided for piercing bone during the implantation process. Grooves or flutes 66 provide space for bone chips to disperse during insertion into locking member 28 and to carry bone debris away from the point 62 as it is progressing through the bone. A suitable structure, such as a hex opening 70 or the like, is provided for engagement of the fastener to apply compressive and/or rotational forces during the implantation process. Depressions or other suitable structure can be provided to permit a fastening device to grip the head 48.

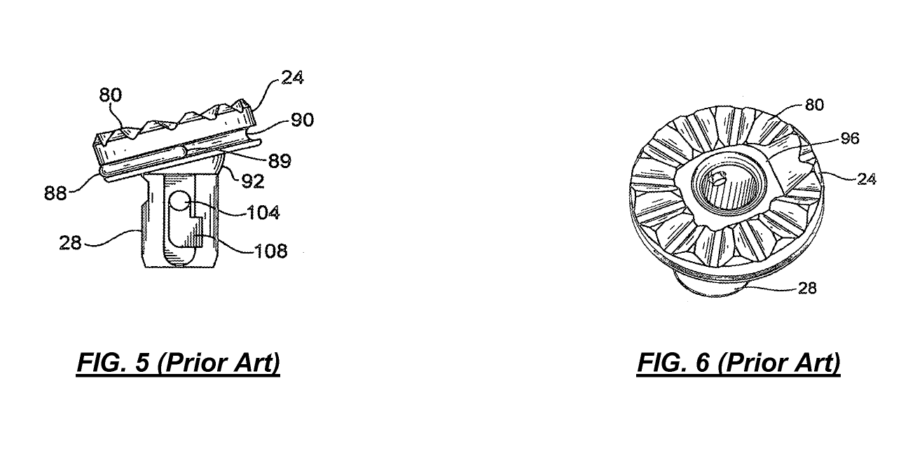

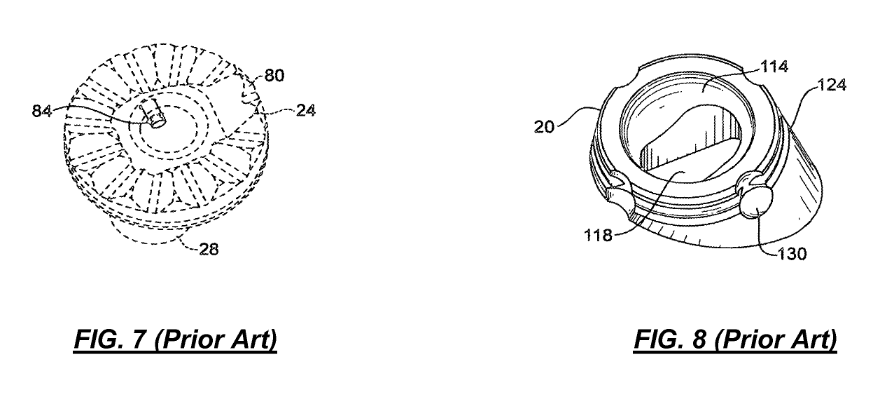

Referring to FIGS. 5-7, the second washer 24 can have any suitable structures, such as serrations 80, for engaging the bone surface. Alternative structures are also possible. The second washer 24 is pivotal with respect to the locking member 28. The locking member 28 can have any suitable structures, such as apertures 84. Cooperating engagement structures, such as a clip 88, can be provided for extending through apertures 89 in the second washer 24 and engaging the apertures 84, such that the second washer 24 engages the locking member 28. A convex surface 92 on the locking member 28 can cooperate with a similar concave surface 96 on the second washer 24. The clip 88 can be positioned in a suitable retaining groove 90 on the second washer 24. An aperture 104 and slotted L-shaped groove 108 can be provided to engage corresponding protrusions on the lower end portion 16 to secure the locking member 28 to the lower end portion 16 of the fastening device 10.

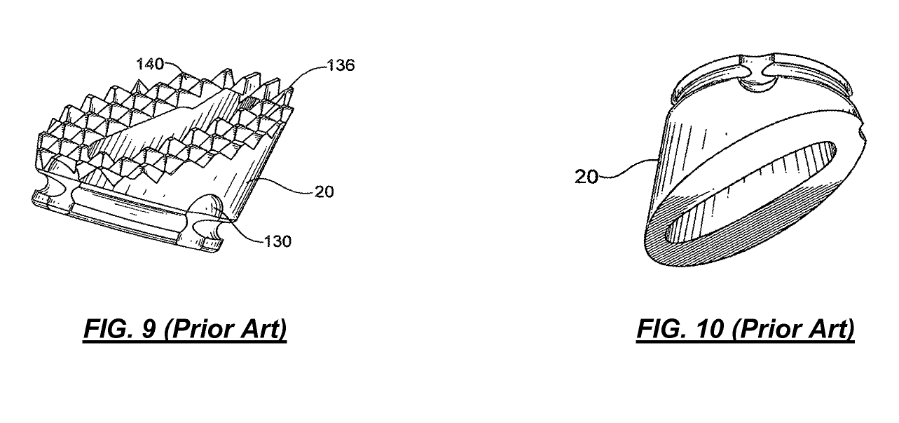

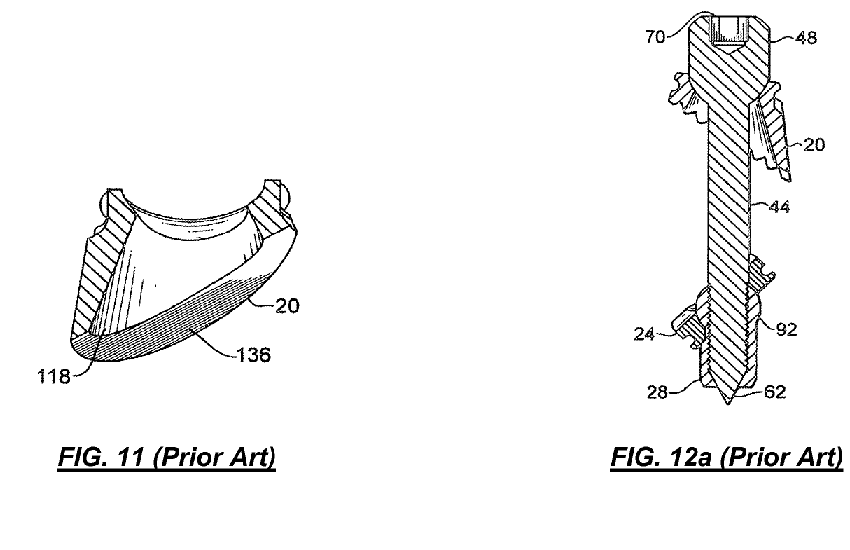

Referring to FIGS. 8-11, the first washer 20 can have a concave surface 114 for cooperating with the convex surface 54 of the head 48 of the fastener 44. An elongated opening 118, which can be in the form of a tapered slot, permits the pivoting of the first washer 20 relative to the fastener 44. The first washer 20 can have any structure for allowing the washer 20 to be engaged by the fastening device 10. This structure can be a circumferential groove 124 which can be engaged by a cooperating flange structure on the fastening device 10. This allows the first washer 20 to be advanced toward the surface of the bone. Additional structures, such as depressions 130, can be provided for engagement by the fastening device 10 to permit rotation of the first washer 20. This assists in properly positioning the first washer 20. The first washer 20 can have an angled contact surface 136, which permits the first washer 20 to cooperate against bone surfaces, such as facets, which present significant angles. Additional structures can be provided to promote engagement. These structures can include serrations 140 on the contact surface 136. The opening 118 can expand wider in a fluted manner toward the contact surface 136 to permit greater pivoting.

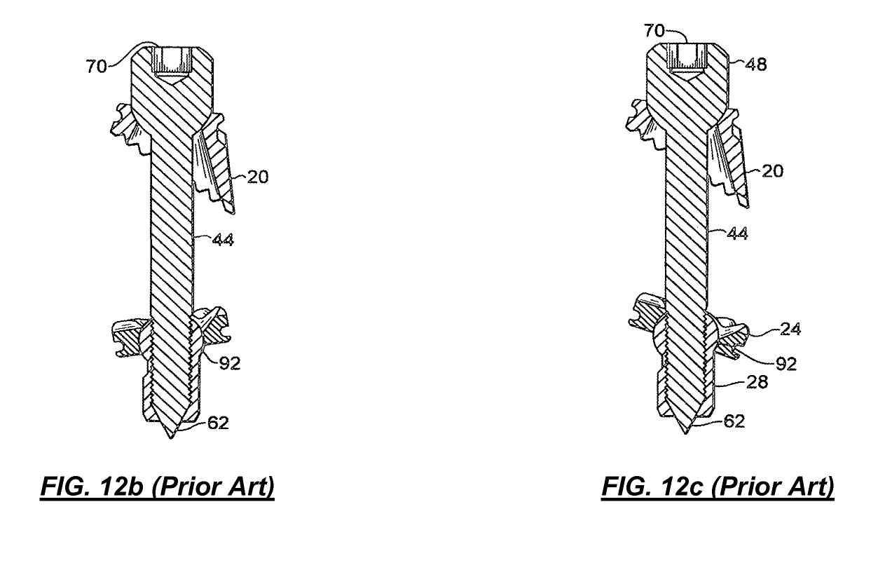

The manner of engagement between the fastener 44, the first washer 20 and second washer 24, and the locking member 28 is illustrated in FIGS. 12a-c. Specifically, the pivoting motion of the first washer 20 is illustrated. This washer can tilt approximately 30° from a transverse section through the screw axis. More or less is possible, depending on the bone system that is being fused. The second washer 24 is capable of tilting approximately +45° to −20° from the transverse section through the screw axis. More or less is again possible.

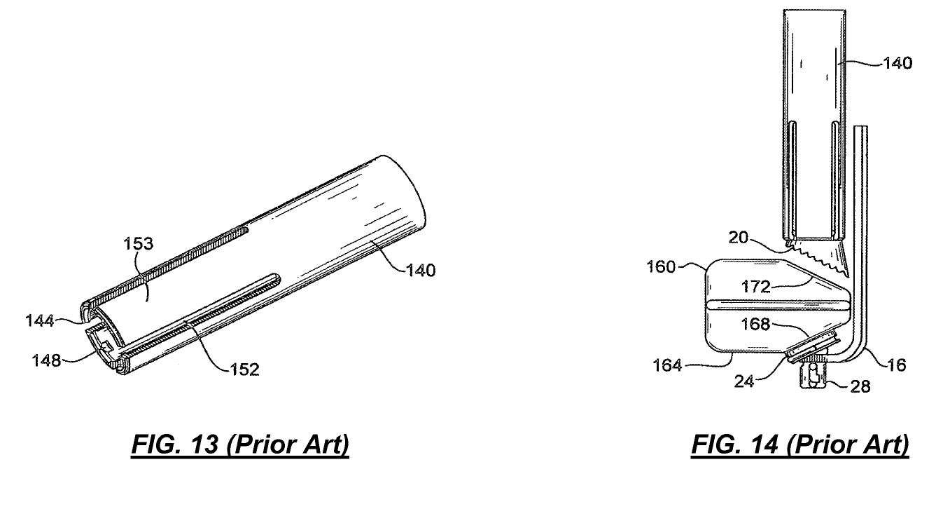

Suitable structures can be provided with the device 10 for engaging the first washer 20. There is illustrated in FIG. 13 a collet 140, although other holding structures are possible. The collet 140 has distal circumferential flanges 144 which engage the groove 124 on the first washer 20. Protrusions 148 are provided to engage the depressions 130 to permit the rotation of the first washer 20. Elongated slots 152 provide leaf springs 153 for creating a spring action on the lower lips 144, such that the collet 140 can engage and disengage from the first washer 20 using moderate manual force.

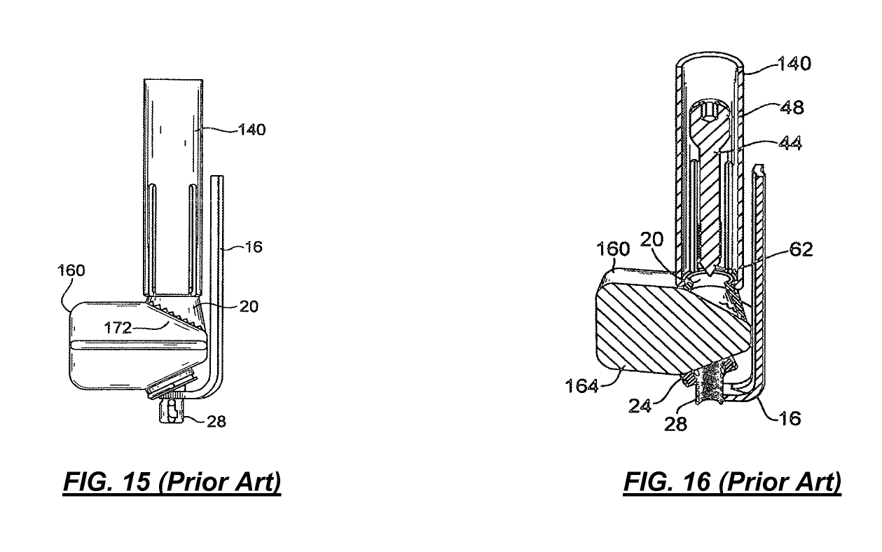

The manner of engaging the first washer 20 and second washer 24 to the bone is illustrated in FIGS. 14 and 15. The locking member 28 is engaged to the lower end portion 16 in a suitable slot. Protrusions in the lower end portion 16 can engage the apertures 104 in the locking member 28. The bone is illustrated schematically as a superior facet 160 and inferior facet 164. The first washer 20 is engaged to the collet 140. The lower end portion 16 is positioned such that the second washer 24 rests against the inferior facet 164. The pivoting of the lower washer 24 relative to the locking member 28 and lower end 16 permits the second washer 24 to match the inclined portion 168 of the inferior facet 164. The collet 140 is then lowered such that the first washer 20 engages the inclined portion 172 of the superior facet 160. The protrusions 148 engage the depressions 130 such that rotation of the collet 140 will rotate the first washer 20 to properly position the first washer 20 relative to the inclined portion 172 of the superior facet 160.

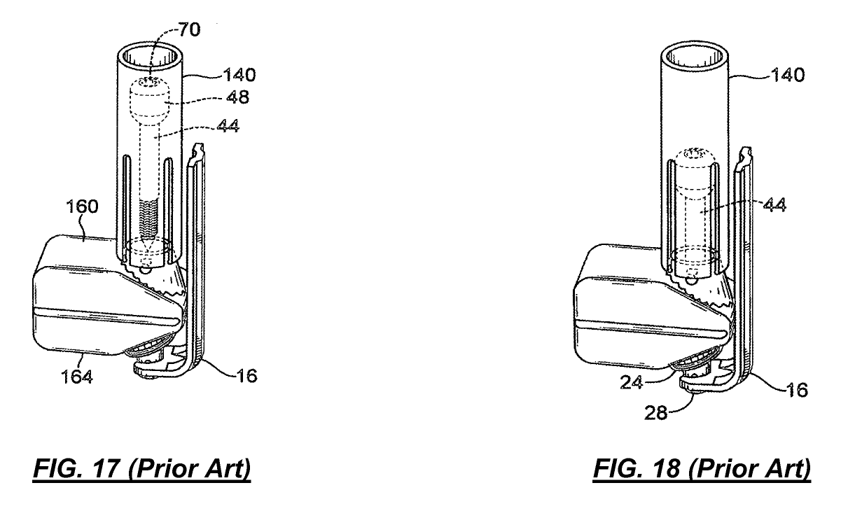

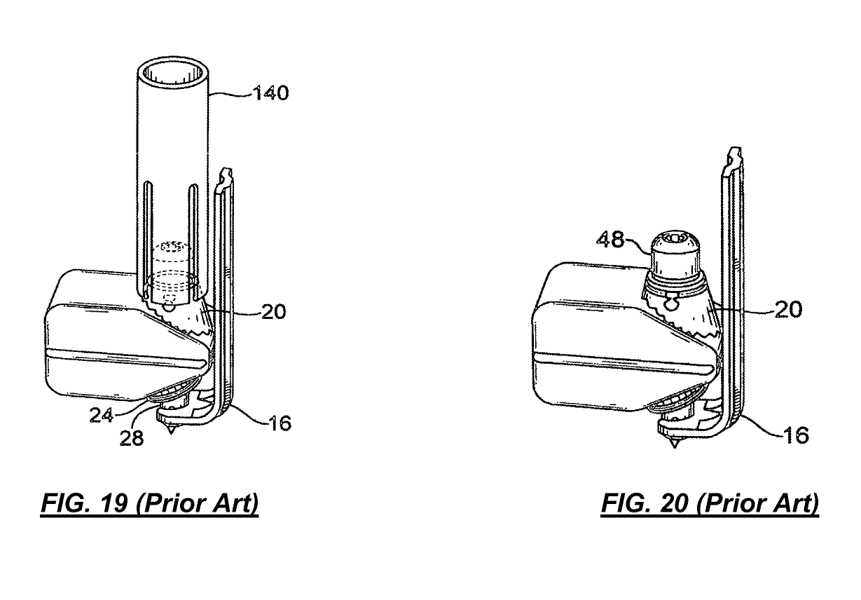

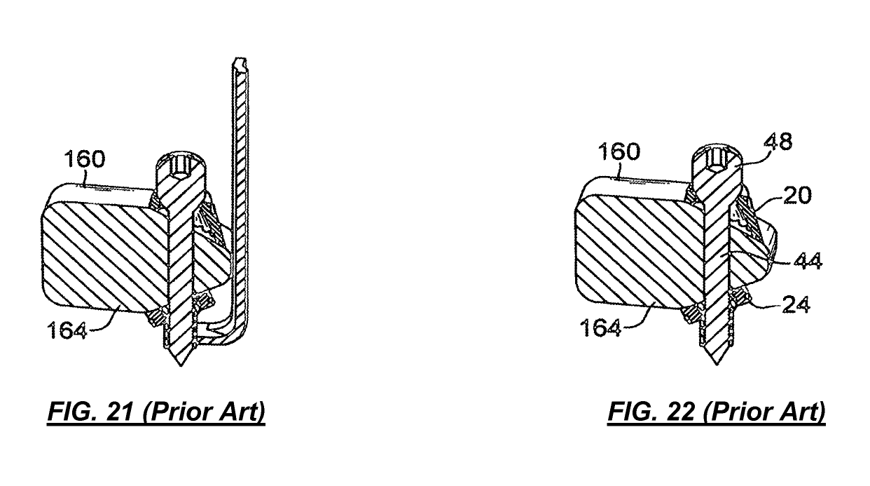

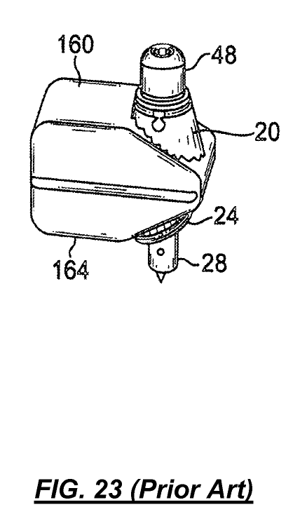

The installation of the fastener 44 is illustrated in the sequence of FIGS. 16-18. The fastener 44 is held by a suitable engagement portion of the device 10 (not illustrated) and driven toward the superior facet 160. The fastener 44 can be engaged such that the head 48 can be rotated and the point 62 can be driven into the facet 160. Flutes can be provided on the pointed end 62 to remove bone debris as the fastener 44 is rotated and pushed into the bone. In this manner, the fastener 44 drills through the superior facet 160 and inferior facet 164. As the fastener 44 is advanced through the bone, it engages the locking member 28. The threads 58 engage female threads in the locking member 28. The fastener 44 is thereby be engaged to the locking member 28, as illustrated in FIG. 19. The collet 140 can then be removed, as illustrated in FIGS. 20 and 21. The lower end portion 16 is then removed as the device 10 is removed, as illustrated in FIG. 22. This results in a completed implant across the superior facet 160 and inferior facet 164, as illustrated in FIG. 23.

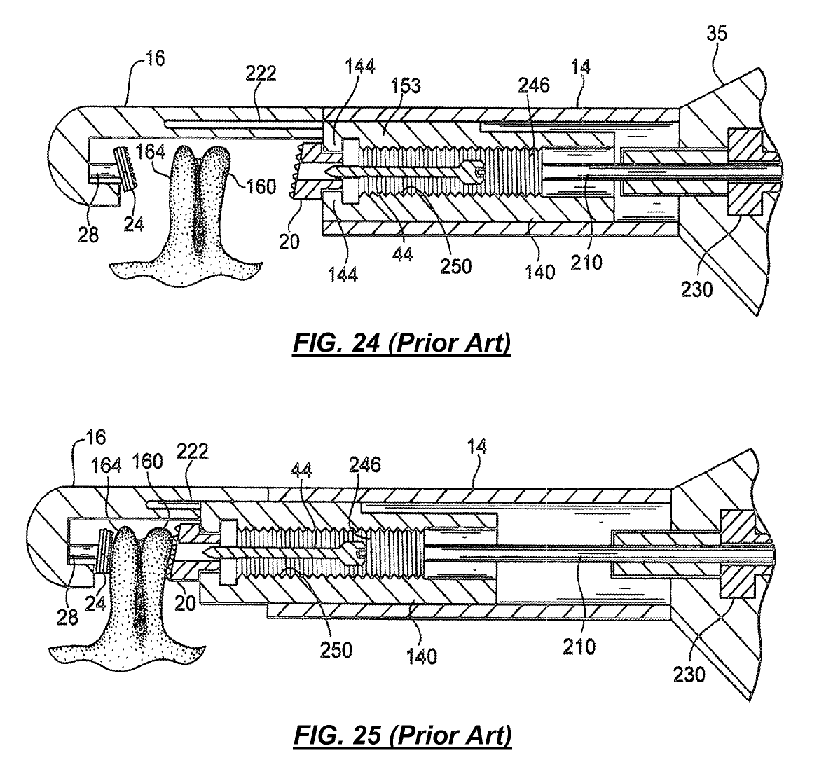

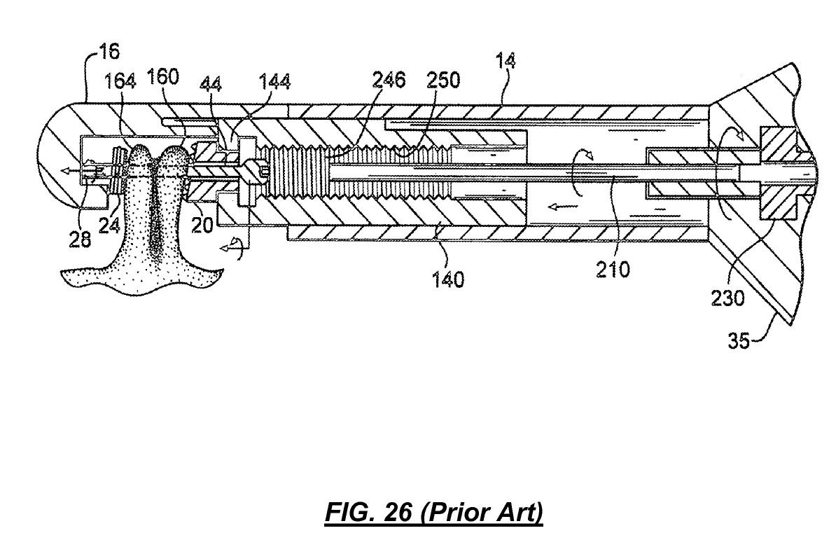

A bone fixation device 10, as used during an implantation procedure is illustrated in FIGS. 24-26. The device 10 has an elongated housing 14 in the general shape of a cannula. Within the housing is the collet 140. The collet 140 has structure for engaging the first washer 20. Any structure is possible, however, the collet 140 in FIG. 24 has inwardly directed circumferential holding flanges 144. The holding flanges 144 engage the groove 124 on the first washer 20. The collet 140 further can have protrusions 148 which engage the depressions 130 in the first washer 20. The first washer 20 is thereby held against movement out of the collet 140 and against rotation relative to the collet 140. The spring force holding the first washer 20 in the collet 140 is such that a manual force can be used to remove the collet 140 from the first washer 20 after the first washer 20 has been secured to the bone.

The collet 140 is mounted in the housing 14 so as to be axially movable therethrough. The collet 140 can have structures for slidably engaging a guide groove 222 or other suitable structures. The first trigger 34 can be operated to move the collet 140 and first washer 20 through the housing 14 from the position illustrated in FIG. 24 to the position illustrated in FIG. 25. When gear 230 is rotated, the shaft 210 is pulled away by the threaded end of the shaft 246. The first washer 20 is seated against the superior facet 160, and the second washer 24 is seated against the inferior facet 164. The guide knob 35 can be rotated to properly position the second washer 24 if the orientation is not correct. The locking lever 32 can be operated to clamp the device to the facet.

The helical gear 230 is then rotated by the action of the trigger 38, which causes the mostly slidable hexagonal or flat faced shaft 210 to rotate. This rotates the extended threaded end of the shaft 246, which is engaged to mating internal threads 250 on an interior surface of the collet 140. The face of threaded end 246 includes structure for engaging the fastener 44, such as a hexagonal tip. Rotation of the threaded end 246 thereby rotates and advances the fastener 44. The fastener 44 advances through the first washer 20, and through the superior facet 160 and inferior facet 164, due to the drilling action created by the forward and rotational movement of the fastener 44. The fastener 44 then advances through the second washer 24 and into the locking member 28. The threads 58 on the fastener 44 engage cooperating threads on an inside surface of the locking member 28. The knob 37 can then be operated to properly torque the implant 40 including to fully seat fastener 44 with locking member 28.

This device 10 provides numerous advantages over the prior art pedicle screw fixation systems. As the bone joint segments, such as the superior facet 160 and inferior facet 164, are compressed between the first washer 20 and second washer 24, there are no internal threads in the bone to raise stresses within the bone. The threads 58 are only on the lower end of the shaft 44 such that these threads engage only the locking member 28 and do not apply thread stresses to the interior of the bone. Also, as the implant is tightened using the rotational force, conventional torqueing mechanisms can be applied such that a known compressive force is applied to the joint. The first washer 20 and second washer 24 can be provided with varied angled contact surfaces to variously fit differing bone geometries for joining bone segments other than the facets. Also, the amount of tilt in the first washer 20 and second washer 24 relative to the fastener 44 can be adjusted depending upon the particular bone geometry that is being fused, owing to the pivotal and polyaxial motion that is permitted. The installation of the implant 40 is reversible. The compression of the implant washers 20 and 24 can be removed to allow repositioning prior to fastener 44 insertion. Accordingly, the device 10 provides great variability and flexibility, in addition to ease, control and consistency of installation.

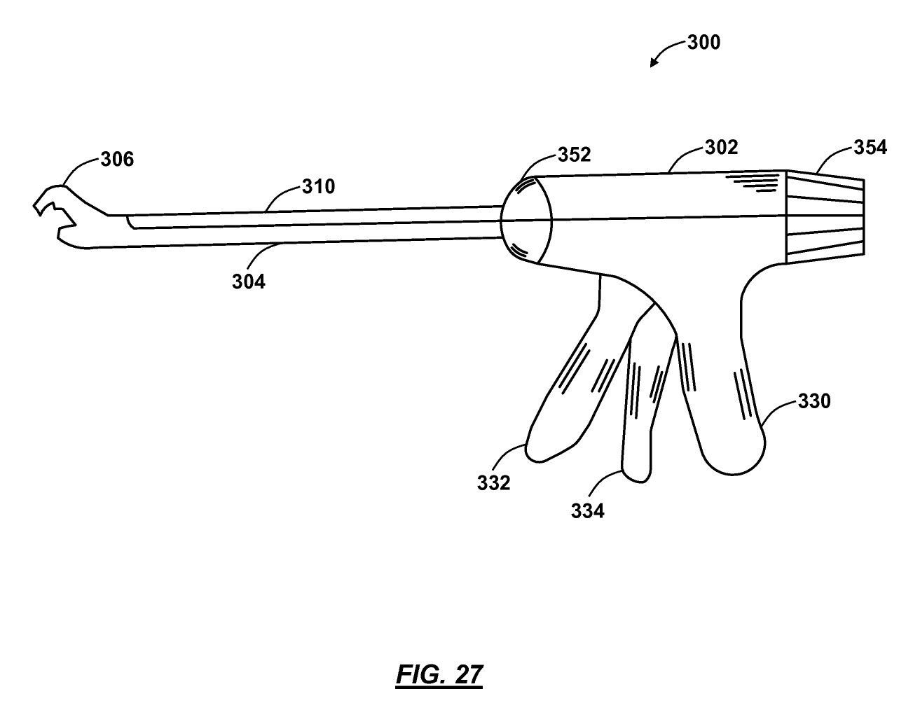

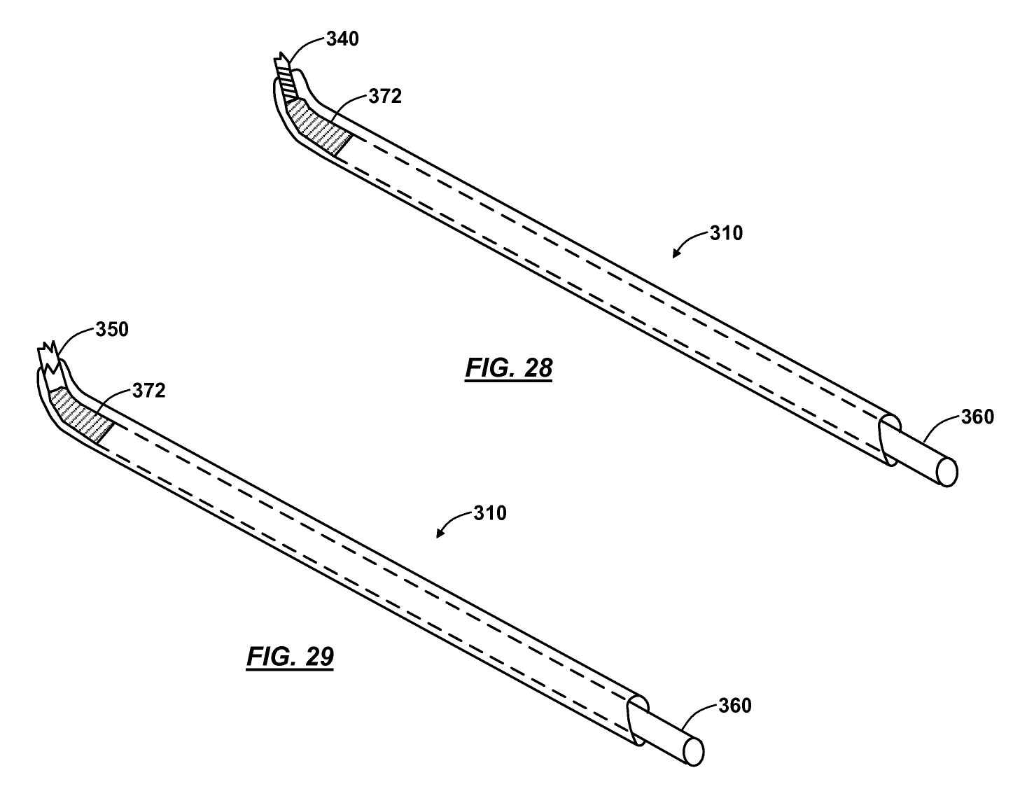

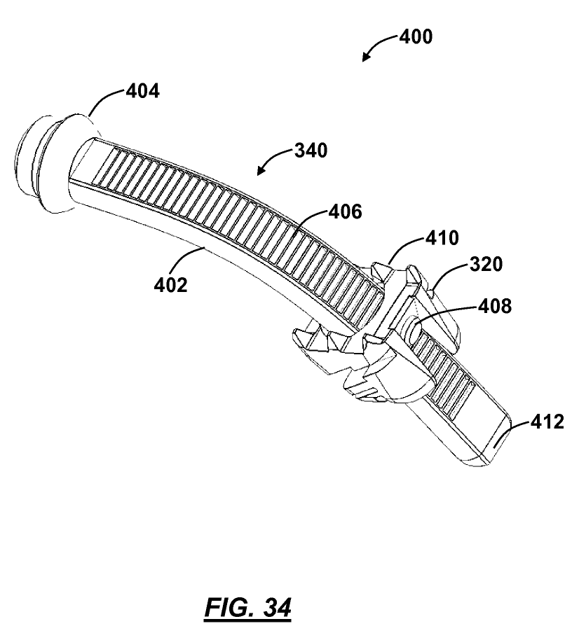

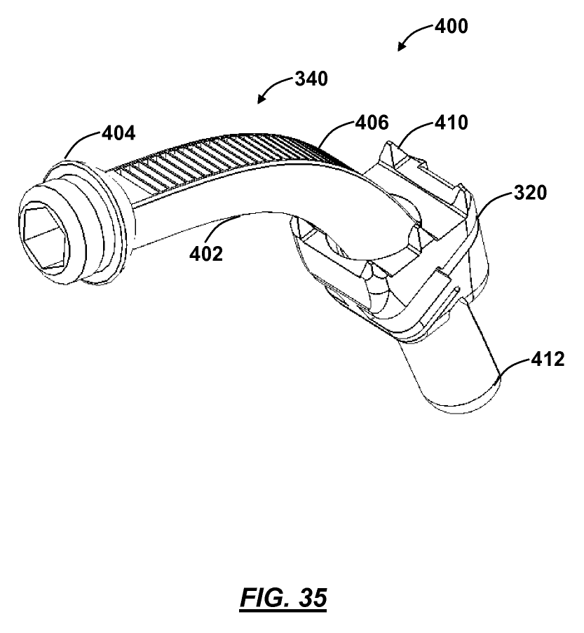

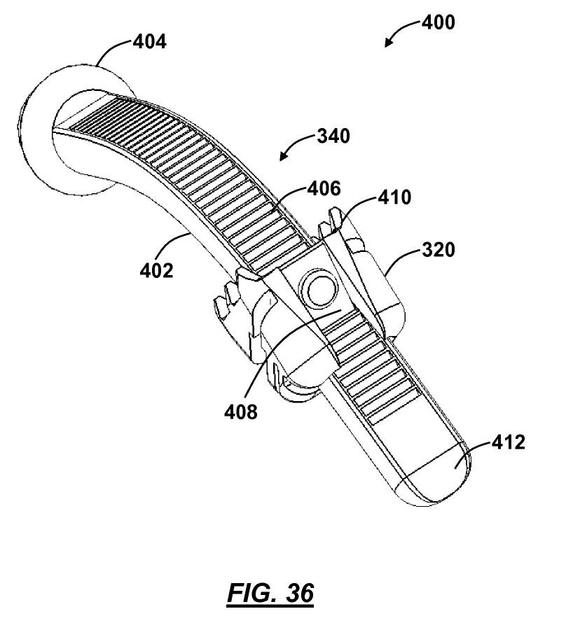

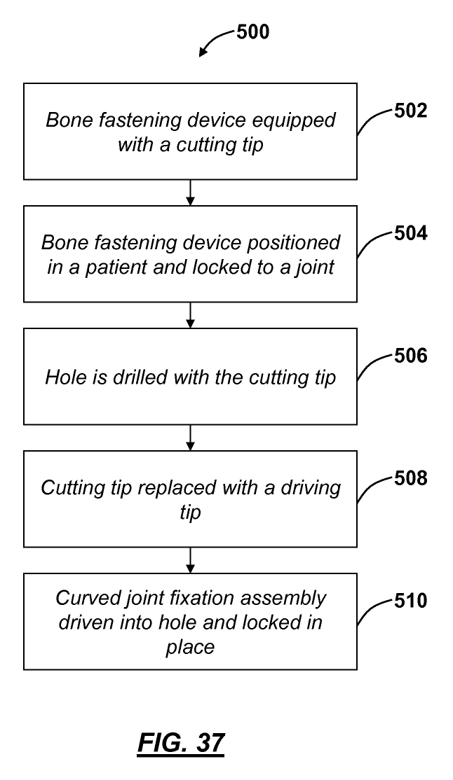

Referring to FIG. 27, a bone fastening device 300 for implantation of a curved facet joint fixation assembly (such as an assembly 400 illustrated in FIGS. 34-36) is illustrated according to an exemplary embodiment of the present invention. The bone fastening device 300 includes a main body 302 and an elongated housing 304 terminating in a lower end portion 306. The elongated housing 304 includes a removable drive 310 (illustrated in FIGS. 28 and 29) within the elongated housing 304 and the main body 302. The bone fastening device 300 has a structure for holding a washer 320 at a distal portion of the lower end portion 306. The lower end portion 306 is curved or at an angle relative to the elongated housing 304 to adapt to various spinal morphologies and to support the curved facet joint fixation assembly during implantation.

A handle 330 is provided to grip the bone fastening device 300, and triggers 332, 334 can be provided to operate the bone fastening device 300 during the implantation process. For example, the trigger 332 can be utilized to lock the bone fastening device 10 to a structure (i.e., a boney structure), and the trigger 334 can be utilized to engage the drive shaft 310 (i.e., to deploy a bolt 340 or a drill head 350).

A guide knob 352 can be operated to rotate the elongated housing 304 and attached lower end portion 304 to properly position the washer 320 and the lower end portion 304. A locking lever (not shown) can be provided to lock the bone fastening device 10 on the bone or joint after the washer 320 has been properly positioned. The locking lever can be unlocked to allow repositioning of the washer 320. A knob 354 can be provided to manually advance the fastener and apply appropriate torque. A slidable protective cannula or sheath can be used to facilitate insertion of the bone fastening device 300 into the body and cover the lower end portion 306 similar to the embodiment described in FIG. 1b.

Referring to FIGS. 28 and 29, the removable drive 310 is illustrated with a bolt 340 and a drill head 350 attached to a drive shaft 360 according to an exemplary embodiment of the present invention. FIG. 28 illustrates the removable drive 310 with the bolt 340 for operation as a driving tip, i.e. to utilize the bone fastening device 300 to drive the bolt 340 into a joint or the like. FIG. 29 illustrates the removable drive with the drill head 350 for operation as a cutting tip, i.e. to cut or bore a hole in a joint prior to receiving the bolt 340. The bone fastening device 300 utilizes a two step implantation process whereby a hole is drilled first with the cutting tip followed by driving the bolt 340 into the hole with the driving tip and locking the washer 320 on the bolt 340.

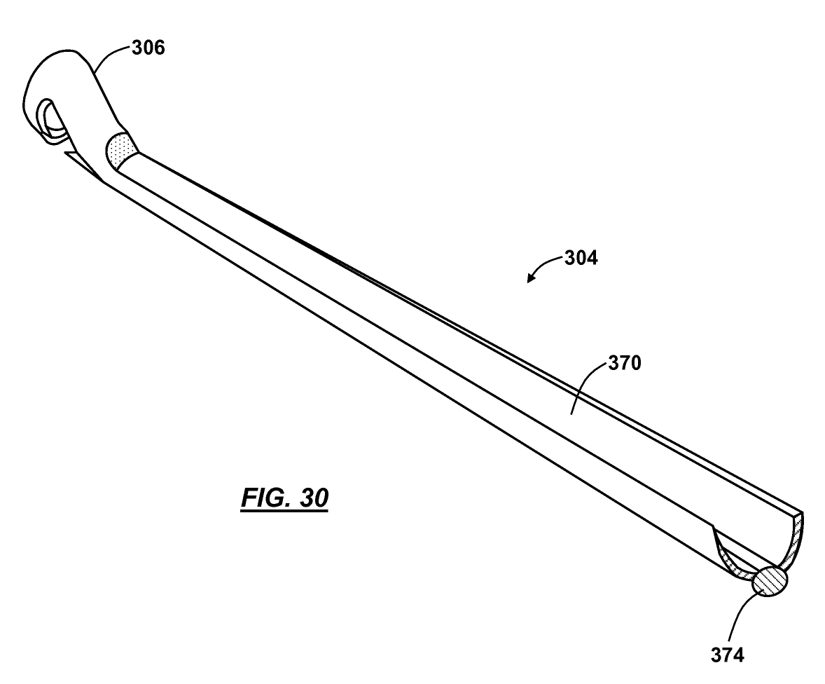

The removable drive 310 fits within an open top 370 (FIG. 30) of the elongated housing 304. The removable drive 310 includes a hollow sheath for receiving the drive shaft 360. The drive shaft 360 includes a flexible drive tip 372 that engages the bolt 340 or the drill head 350. The flexible drive tip 372 includes a flexible area within the angled portion of the lower end portion 306. The drive shaft 310 is operable to provide torque and an angled force to either the bolt 340 or the drill head 350 with the torque or the force translated through the flexible drive tip 372. The flexible drive tip 372 can include a coating to lessen friction in the angled portion of the lower end portion 306. Additionally, the flexible drive tip 372 can move or flex when inserting the removable drive 310 in the open top 370 (FIG. 30) of the elongated housing 304. The present invention contemplates various lengths for the drive shaft 360, and the opposite end of the drive shaft 360 relative to the flexible drive tip 372 is configured to receive torque through various mechanisms, such as a drill. For example, the bone fastening device 300 can include an opening in an end of the knob 354 to receive the drill or the like. In an exemplary embodiment, the removable drive 310 is disposable after each use.

Referring to FIG. 30, the elongated housing 304 is illustrated with the removable drive 310 removed showing the open top 370 according to an exemplary embodiment of the present invention. As described herein, the removable drive 310 can be placed within the bone fastening device 300 through the open top 370 in the elongated housing 304. The open top 370 allows the angled removable drive 310 to fit within the lower end portion 306 by flexing the flexible drive tip 372. A fixation tong 374 is located at an end of the lower end portion 306.

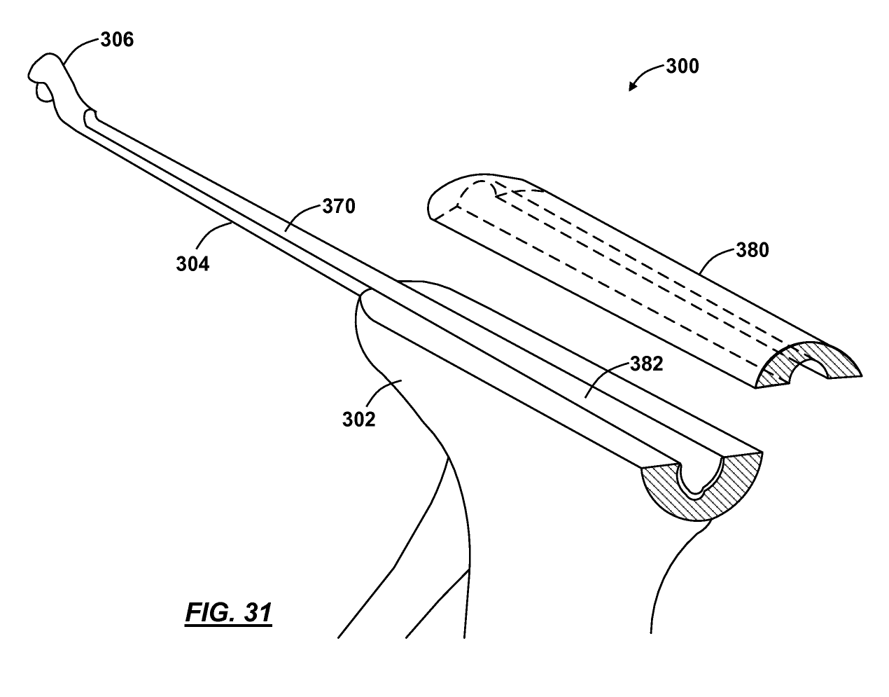

Referring to FIG. 31, the bone fastening device 300 is illustrated with the removable drive 310 removed according to an exemplary embodiment of the present invention. The main body 302 includes a removable top 380 that can be removed to place the removable drive 310. When attached, the removable top 380 and the main body 302 include a cylindrical channel 382 where the removable drive 310 extends through the main body 302. The cylindrical channel 382 extends to an end of the main body 302 where a torque mechanism can be applied to the drive shaft 360. The removable top 380 can be snapped, latched, locked, etc. in place to the main body 302. Optionally, the removable top 380 can be integrated with the removable drive 310 in a single device.

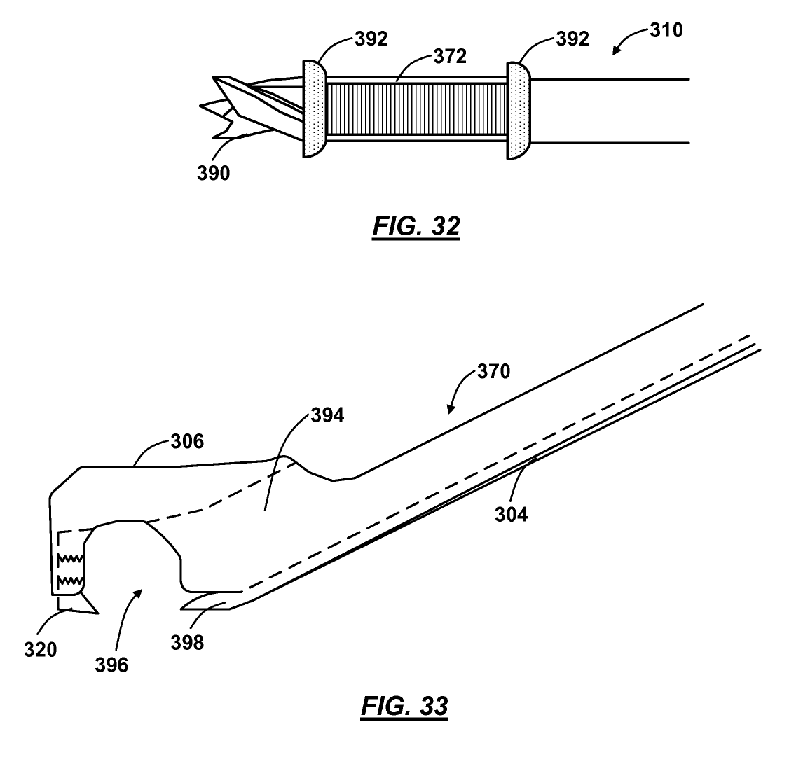

Referring to FIG. 32, a top view illustrates a tip of the removable drive 310 with a chisel tip bit 390 for operating as a cutting tip according to an exemplary embodiment of the present invention. As described herein, the flexible tip 372 is operable to fit the angle of the lower end portion 306. The removable drive 310 can include stoppers 392 at each end of the flexible tip 372. The stoppers 392 can be coated in a flexible material such as poly(tetrafluoroethylene) or poly(tetrafluoroethene) (PTFE) (i.e., Teflon® available from DuPont). The stoppers 392 are dimensioned to rotatably fit and slide with reduced friction within a cylindrical portion 394 of the lower end portion 306. The flexible tip 372 is constructed to both flex and translate rotational torque and a driving force (based on whether the removable drive 310 is used as a cutting or a driving tip).

Referring to FIG. 33, a cross-sectional view illustrates the lower end portion 306 with the removable drive 310 removed according to an exemplary embodiment of the present invention. The lower end portion 306 is at an angle relative to the elongated housing 304 to adapt to various spinal morphologies and to support the curved facet joint assembly. In an exemplary embodiment, the angle can be between 10 and 60 degrees. The lower end portion 306 includes an opening 396 where a joint (e.g., a facet joint) is placed and locked in place. When engaged, the removable drive 310 extends from the elongated housing 304 through the cylindrical portion 394 to the opening 396. At the opening 396, the removable drive 310 can be utilized as a cutting tip to provide a hole in the joint and as a driving tip to drive a bolt through the hole. At a distal end of the opening 396 on the lower end portion 306, the washer 320 is held in place to engage the bolt 340. The washer 320 can include a plurality of barbs or a pawl for setting and locking a position on the bolt 340.

The lower end portion 306 can include a locking mechanism 398 operable to engage a joint and maintain a position on the joint. Specifically, the lower end portion 306 can remain locked while the removable drive 310 is utilized and replaced, i.e. to switched between a cutting tip and a driving tip.

Referring to FIGS. 34-36, various perspective views illustrate a curved facet joint fixation assembly 400 for implantation in a spine or the like according to an exemplary embodiment of the present invention. The curved facet joint fixation assembly 400 includes the bolt 340 and the washer 320. The bolt 340 includes an elongated, curved shaft 402 and a head 404. The elongated, curved shaft 402 engages the washer 320, and the elongated, curved shaft 402 includes a plurality of ridges 406 for engaging a pawl 408 on the washer 320. The plurality of ridges 406 and the pawl 408 enable the washer 320 to move towards the head 404 along the elongated, curved shaft 402 only. Other engagement structures can also be used. Accordingly, the bone fastening device 300 can be used (with the trigger) to drive the washer 320 onto the bolt 340 and to lock the washer 320 in an appropriate position.

The head 404 is illustrated with a hexagonal opening for engaging the removable drive 310. For example, the removable drive 310 can include a hexagonal structure to engage the head 404. Other engagement structures can also be used. The washer 320 can have any suitable structures, such as serrations 410, for engaging the bone surface. Alternative structures are also possible. The elongated, curved shaft 402 terminates in a rounded end 412. The curved facet joint fixation assembly 400 requires a hole to be predrilled prior to implantation, such as through the cutting tip described herein.

The curvature of the curved facet joint fixation assembly 400 is adapted to fit spinal morphologies. The present invention contemplates a variety of curvatures and lengths for the curved facet joint fixation assembly 400 to fit a variety of spinal morphologies. For example, the curved facet joint fixation assembly 400 can come in a variety of lengths and curvatures. A surgeon can select the specific length and curvature based on the actual morphology of a joint receiving the curved facet joint fixation assembly 400,

Referring to FIG. 37, a flowchart illustrates a curved facet joint fixation assembly implantation mechanism 500 according to an exemplary embodiment of the present invention. First, a surgeon provides an opening in a patient, such as through minimally invasive surgery (MIS). A bone fastening device is equipped with a cutting tip (step 502). The bone fastening device is positioned in the patient and locked to a joint (step 504). The cutting tip is utilized to bore or drill a hole through the joint (step 506). The cutting tip is replaced with a driving tip (step 508). A curved joint fixation assembly is driven into the hole and locked in place (step 510).

As described herein, the implantation mechanism 500 can utilize the bone fastening device 300. With the implantation mechanism 500, the bone fastening device 300 can lock in place initially with the cutting tip to bore the hole. Once the hole is formed, the bone fastening device 300 can remain locked in place while the cutting tip is replaced with the driving tip. Once replaced, the curved joint fixation assembly can be driven into the hole and locked in place by pulling the trigger thereby positioning the washer in place.

The various components above are constructed with constructions of surgical grade plastics or metals, such as titanium. Different dimensions of the various components are within the spirit and scope of the present invention.

Although the present invention is illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the present invention, are contemplated thereby, and are intended to be covered by the following claims.

What is claimed is:

1. A curved joint fixation assembly, comprising:
a curved elongated shaft comprising a head and an end; and
a washer having a structure for engaging the end;
wherein the curved elongated shaft is curved based on a spinal morphology of a receiving patient.

2. The curved joint fixation assembly of claim 1, further comprising:
a plurality of ridges on the curved elongated shaft; and
a pawl on the washer for engaging the plurality of ridges.

3. The curved joint fixation assembly of claim 1, wherein the head comprises a hexagonal opening connected to a removable drive of a bone fastening device.

4. The curved joint fixation assembly of claim 1, wherein the end comprises a rounded surface.

5. The curved joint fixation assembly of claim 1, further comprising:
a plurality of serrations on the washer for engaging a bone surface.

6. The curved joint fixation assembly of claim 1, wherein the curved elongated shaft is disposed to a removable drive of a bone fastening device, and wherein the washer is disposed to a lower end portion of the bone fastening device.

7. The curved joint fixation assembly of claim 6, wherein the bone fastening device comprises a cutting tip to bore a hole in a joint and a driving tip to drive and lock the curved joint fixation assembly in the hole.

8. A curved joint fixation assembly implantation tool, comprising:
an elongated housing terminating in a lower end portion, wherein the lower end portion is at an angle in relation to the elongated housing;
a removable drive disposed within the elongated housing, wherein the removable drive terminates with one of a cutting tip and a driving tip; and
a curved joint fixation assembly comprising a curved elongated shaft disposed to the driving tip and a washer disposed to the lower end portion.

9. The curved joint fixation assembly implantation tool of claim 8, wherein the curved elongated shaft is curved based on a spinal morphology of a receiving patient.

10. The curved joint fixation assembly implantation tool of claim 8, wherein the removable drive comprises a flexible tip adapted to fit within an opening from the elongated housing to the lower end portion.

11. The curved joint fixation assembly implantation tool of claim 10, wherein the flexible tip comprises a coating of a flexible material.

12. The curved joint fixation assembly implantation tool of claim 8, further comprising:
a main body disposed to the elongated housing;
wherein the main body comprises a handle and one or more triggers.

13. The curved joint fixation assembly implantation tool of claim 8, further comprising:
an opening in the main body for providing torque to a drive shaft in the removable drive.

14. The curved joint fixation assembly implantation tool of claim 8, wherein the removable drive comprises a first removable drive comprising a cutting tip, and further comprising a second removable drive comprising a driving tip with the curved joint fixation assembly.

15. The curved joint fixation assembly implantation tool of claim 14, wherein the first removable drive is utilized to bore a hole in a joint and removed from the elongated housing and replaced with the second removable drive to drive in the curved joint fixation assembly.

16. A method of implanting a curved joint fixation assembly, comprising:
boring a hole in a joint with a cutting tip;
removing the cutting tip;
positioning a driving tip comprising a curved joint fixation assembly;
driving in the curved joint fixation assembly; and
locking the curved joint fixation assembly.

17. The method of claim 16, further comprising:
determining a spinal morphology of a receiving patient; and
selecting a curvature of the curved joint fixation assembly responsive to the spinal morphology.

18. The method of claim 16, wherein the method is performed by a bone fastening device.

19. The method of claim 18, wherein the bone fastening device comprises:
an elongated housing terminating in a lower end portion, wherein the lower end portion is at an angle in relation to the elongated housing;
a removable drive disposed within the elongated housing, wherein the removable drive terminates with one of the cutting tip and the driving tip; and
a curved joint fixation assembly comprising a curved elongated shaft disposed to the driving tip and a washer disposed to the lower end portion.

20. The method of claim 19, further comprising:
locking the bone fastening device to the joint; and
performing the removing and positioning steps while the bone fastening device is locked to the joint.
